(19) United States
(12) Patent Application Publication
(10) Pub. No.: US 2026/0345876 A1
(43) Pub. Date: Nov. 5, 2026

(54) Method to Control a Breast Pump, Breast Pump and Computer Program Code

(71) Applicant: MEDELA AG, Baar (CH)

(72) Inventor: Carsten Faltum, Vitznau (CH)

(21) Appl. No.: 19/121,307

(22) PCT Filed: Oct. 17, 2022
(86) PCT No.: PCT/EP2022/078830

§ 371 Date: Apr. 15, 2025

(51) Int. Cl. A61M 1/06

(52) U.S. Cl. CPC A61M 1/06; A61M 2202/0007; A61M 2202/04; A61M 2205/3331; A61M 2205/3379; A61M 2205/3592; A61M 2205/505; A61M 2210/1007

(57) ABSTRACT

A method to control a breast pump having a suction source, connection means and a first control device, the first control device is adapted to control the suction source, and the first control device is connected via the connection means to a second control device which is a hand held device separate from the breast pump, the method including
providing at least two control presets (PR1, PR2, PR3, PR4), each of the at least two control presets (PR1, PR2, PR3, PR4) including one of at least two control parameters (P1, P2, P3) intended for control of the first control device,
selecting one control preset (PR1, PR2, PR3, PR4) of the at least two control presets (PR1, PR2, PR3, PR4) as the selected control preset (PR-SEL),
sending the control parameter (P1, P2, P3, P4) of the selected control preset (PR-SEL) to the first control device and controlling the first control device,
the selected control preset (PR-SEL) is selected through a position of a throttle. Additionally, a breast pump, a computer program code to perform the method and a second control device running the computer program code.

The present invention relates a method to control a breast pump having a suction source, connection means and a first control device, wherein the first control device is adapted to control the suction source, and wherein the first control device is connected via the connection means to a second control device which is a hand held device separate from the breast pump. The present invention further relates to a breast pump, to a computer program code and to a second control device.

A breast pump with a first and a second control device is known from WO 2018/229504 A 1. The breast pump is used to express milk from a human breast by means of a vacuum/suction that is applied in cycles. The end user may control the breast pump on the second control device in form of a hand held device such as a smartphone in order to express milk. In WO 2018/229504 A1 the end user is able to manually control breast pumps individually with the help of a visualized control panel (see FIG. 21 in the prior art) and by a selection of control presets, i.e. start, stop or change a pumping cycle, increase or decrease each pump peak pressure, or switch between different pre-program pressure profiles such as one mimicking the sucking pattern of a baby during an expression or stimulation cycle.

In said prior art the end user needs a basic knowledge as to what control presets shall be chosen to get good milk expression results and to have the best comfort and/or little to no pain during expression. This for example necessitates trial and error use of the breast pump.

It is an object of the present invention to provide a method to control a breast pump and an according breast pump that can be used with less basic knowledge and/or more intuitively. In particular, a breast pump and a method to control shall be provided that can be used so as to generally improve the end user's comfort during the expression of milk.

As a solution to this problem, the present invention provides a method according to the features of claim 1. Another solution is provided by the breast pump with the features of claim 10, by the computer program code with the features of claim 13 and by the second control device with the features of claim 14. Preferred embodiments are given in the dependent claims.

Suggested is a method to control a breast pump having a suction source, connection means and a first control device, wherein the first control device is adapted to control the suction source, and wherein the first control device is connected via the connection means to a second control device which is a hand held device separate from the breast pump, the method including the steps of
providing on the second control device at least two control presets, each of the at least two control presets including one of at least two control parameters intended for control of the first control device,
selecting on the second control device one control preset of the at least two control presets as the selected control preset,
sending by means of the second control device the control parameter of the selected control preset to the first control device and controlling the first control device by means of said control parameter.

According to the invention, the selected control preset is selected through a position of a throttle which is slidable on a surface of a visualized control panel of the second control device to define the position of the throttle. In other words, the second control device may display the control panel including the throttle that is movable along the surface, for example back and forth along one direction, typically with the help of the end user's finger tip. Upon movement of the throttle to another specifically linear or rotational position, a control preset is selected from at least two control presets defining the selected control preset. The position of the throttle may continuously be visualized on the breast pump and/or a visualized control panel of the second control device.

The present invention provides the advantage that the method can be conducted more user-friendly. The control may easily be implemented via apps on a smartphone. The possibility to control via a hand held device such as a smartphone contributes to mother's comfort because commonly she is used to using a smartphone and because of the self-explanatory visualization of the control as to what control preset is selected. In particular, the throttle is self explanatory in that it is inherently clear to the end user what the min/max threshold of the presently adjusted kind of control preset is. The throttle can be adjusted in discrete steps or continuously, the later allow-ing the user to select any value intermediate of a maximum and a minimum value of a control parameter and/or control preset. From the throttle it is unambiguously visible which control preset is chosen in what range, since the scale of the throttle is inherently given/visualized. This is an advancement relative to the prior art since the end user may due to the invention more easily remember from visual memory what her most comfortable and/or successful control preset is. In addition, in the surface of the control panel information regarding a better position of the throttle may very easily be implemented, so that the end user can compare his choice quickly to the better position.

The breast pump may comprise a pump housing. The suction source may comprise an internal suction source of the breast pump, for example contained within and/or attached to the pump housing. The suction source may as well contain and/or be connectable to an external suction source, for example which is connected to the pump housing via a suction source interface. In other words, the source of vacuum may be integrated into the pump housing or may be provided externally, either as a part of the breast pump or as a separate part to be connectable to the breast pump.

The throttle may be a slider. The throttle may be arranged slidable along the surface of the control panel. The throttle may comprise a round visualized form or a rectangular visualized form.

The control panel may have one or two dimensions and the throttle is movable along the one or two dimensions to define the position of the throttle. The throttle may be movable linearly, i.e. along one direction. The throttle may also be movable along two perpendicular directions. This opens up the possibility to present a variety of control presets to the end user, for example more than five, more than ten or more than fifty control presets. It may also be possible that the throttle is movable without steps. It may be interpolated between control presets if the throttle is positioned between control presets.

The selected control preset and/or the position of the throttle may further be selected through a continuous control mode acting on the second control device, the continuous control mode being able to select different control presets over time. It can be provided that there is an option on the second control device that activates the continuous control mode, for example a visualized button or a checkbox. The continuous control mode can be understood as an automatic and/or autonomous mode, where the end user can free herself from deciding which control preset to select. The continuous control mode may consider data from the first control device in order to select the control preset. The continuous control mode may for example correspond to a pattern of on/off cycles for the suction source that changes over time.

The at least two control parameters may correspond to and/or contain physical values. The at least two control parameters may include at least one of a pump duration, pump amplitude, a pump on-time, a pump off-time, a pump frequency, a milk volume, a number of pump sessions, and a position of the breast pump. Each control parameter and/or control preset may be displayed individually and exclusively on the visualized control panel of the second control device together with a throttle assigned to said one control parameter and/or control preset. Plural control parameters and/or control presets may be selected on the visualized control panel of the second control device to be displayed sequentially either individually, i.e. as the only control parameter (and/or control preset) at one time and with the assigned throttle, or may be displayed simultaneously with a single throttle assigned to each of the one other or plural other control parameters (and/or control presets).

The control preset may be labeled with the control parameter it contains, for example “pump frequency X” or “pump frequency Y”. The control preset may as well be labeled with a less objective content such as “weak stimulation” or “strong stimulation”. As such, the end user may understand more from the label “weak stimulation” than “pump frequency X” which facilitates that the basic knowledge for using the breast pump merely needs to be little.

The method may comprise the step of providing on the second control device a suggested control preset as one of the at least two control presets and visualizing the suggested control preset on the second control device. This particularly means that on the second control device and/or the surface of the control panel there may be provided a suggestion for the user. The user may adopt the suggestions as the selected control preset instead of the presently selected control preset, that the end user likely had selected once before. It may be visualized another position of the throttle corresponding to the suggested control preset, e.g. by another throttle that is faded out, dotted, highlighted, etc. The suggested control preset may be a suggested position of the throttle. This facilitates an easy use of the breast pump, especially with little basic knowledge.

The suggested control preset may be provided by the first control device or the second control device, especially by means of the continuous control mode. The second control device may consider data from the first control device in order to provide the suggested control preset.

The second control device may serve as a display, in particular for the suggested control preset. The first control device may serve to directly control the suction source and in particular to provide the suggested control preset and/or data to the second control device. Therefore, the breast pump may be technically functioning independent from the second control device, so that the probability of malfunction is reduced.

The method may comprise the step of recording of pump data as recorded pump data when the breast pump is running. The recorded pump data may include at least one of a pump duration, pump amplitude, a pump on-time, a pump off-time, a pump frequency, a milk volume, a number of pump sessions, and a position of the breast pump. The recorded pump data may comprise certain data as a function of the time. The recorded pump data may comprise aggregated values. The presence of recorded pump data serves the purpose to give feedback to the end user, especially on the second control device. For example, the end user may look up the volume of expressed milk in one particular session, on one particular day and/or per particular breast. The recorded pump data may as well serve to provide a suggested control preset, for example to suggest to turn off the breast pump or to decrease the pump amplitude for health reasons.

The recording may be conducted by the second control device. This facilitates a retrofit to the breast pump.

The second control device may be designed to store the recorded data and/or equipped with a storage for the recorded data. The second control device may send the recorded data to a cloud service. The cloud service may send feedback to the second control device, for example provide cloud-based suggested control presets. This as well enables that the second control device may handle the recorded data independent from the first control device. For example, the end user may review the data when the first control device is disconnected.

The suggested control preset may be determined based on the recorded pump data. Not only can the current pump data help to suggest a control preset, but also the recorded pump data. In this regard, the first and/or second control device may learn individual reactions of the end user to an adaption of the selected control preset which helps to suggest a control preset better.

According to another solution of the invention there is provided a breast pump to perform the method as described herein, the breast pump having a suction source, connection means and a first control device, wherein the first control device is adapted to control the suction source, and wherein the first control device is adapted to be connected via the connection means to a second control device which is a hand held device separate from the breast pump.

The connection means may comprise a wired and/or a wireless interface. For example, a mini/micro USB, USB C or other wired interface may be provided. A bluetooth or a wifi interface may be provided. Such interfaces enable a reliable and universal connection for a variety of second control devices and/or charging devices for a battery/an accumulator in the breast pump.

In the following, preferred features and embodiments of the breast pump that work well with the method and particularly the computer program code are described. In particular, a breast pump shaped at least in part to fit inside a bra, especially with an integrated suction source and with an integrated milk container that as well fit at least in part inside the bra, is desired. Hence, the following features at in principle beneficial to the end user comfort in that the breast pump may better fit in the braw and/or that fewer external devices/parts aside from the second control device are necessary to perform the method.

The breast pump may comprise a housing. In particular, a milk container detachably supported by the housing may be provided. The housing may hold the suction source, the connection means and/or the first control device. This reduces the size of the breast pump and facilitates an easy use.

The breast pump housing may comprise and/or contain the suction source and/or the first control device. The breast pump and/or the breast pump housing may comprise a digital display and/or at least one button, particularly a button adapted to bring the connection means into a pairing mode upon pressing the button.

The breast pump may comprise means to acquire pump data, for example means for monitoring milk flow and/or pumping performance including proper alignment of the nipple within the nipple tunnel. A camera can for example be integrated into the breast pump, preferably within the housing, which camera may be connected to a mobile device like a mobile phone to allow visual monitoring via the screen of the handheld device/mobile phone, which screen defines a visualized control panel. The breast pump can for example integrate a sensor as described in EP 3 373 997 B1, WO 2019/145163 A1 or WO 2018/210685A1. The camera mentioned above is preferably received within the housing. The camera shall be adapted to observe at least a portion of the nipple tunnel, which portion is essential for checking proper nipple alignment within the nipple tunnel and/or checking milk flow through or out of the nipple tunnel.

The breast pump should be shaped at least in part to fit inside a bra. The breast pump may comprise a breast shield element which may include a nipple tunnel adapted to receive a nipple. The breast shield element and the milk container may be detachable from the housing. The breast shield element, especially the nipple tunnel, may be connected to the milk container. In principle, the housing may be placed between the milk container and the breast shield element and/or the nipple tunnel may pass through the housing. This facilitates a compact breast pump that may fit into a bra. This also facilitates hygiene of the breast pump. In particular, in combination with the second control device the end user is faced with fewer devices that need to be handled.

The suction source may be of a membrane pump type which may essentially be fully integrated into the housing.

Generally speaking, it is preferred that vacuum/suction is physically generated within the housing. In this regard the housing may hold a rechargeable battery/accumulator, in particular adapted to energize the suction source and/or the first control device. This particularly helps for in-bra use.

The suction source may have a protective membrane element to cover an aggregate membrane that may be moved by turning a spindle of the suction source. The suction source may be built so that when the aggregate membrane is moved a membrane working chamber particularly in front of the protective membrane element may be changed in its volume to have the possibility for a suction function.

The housing may be made of opaque and/or transparent plastic material. In a side view, the housing may be wedge-shaped. A bottom section of the housing may be considerably thicker than a top section of the housing. In a front or rear view, the housing may be essentially avocado-shaped and/or round. This as well helps for in-bra use by improving end user comfort.

The housing may circumferentially enclose a bore to insert the breast shield element.

The milk container may be detachably attached to the housing.

The milk container typically has a milk chamber in order to store expressed milk.

When the breast shield element is inserted through the bore, its nipple tunnel may be connected to the milk container via a thread of the nipple tunnel to a thread of the milk container. Hence, the nipple tunnel may project through the bore.

A flange of the breast shield element may receive a nipple and/or an areola.

The milk container may comprise a in particular spherical container shell element and in particular a lid element closing the container shell element.

The lid element may be connected with the container shell element by means of a hinge. Both the lid element and the spherical container shell element may provide surfaces defining the hinge. Cooperating surfaces of the hinge may allow pivoting of the lid element toward the container shell element.

Proper alignment of a nipple may be observed through a transparent window in the milk container, e.g. from above, from a side and/or from beneath.

The suction source may be activated, for example upon pressing a button e.g. above on the housing giving a command to the first control device which then controls the suction source and/or upon an interaction on the second control device by the end user which then sends a control parameter to the first control device which then controls the suction source.

When the suction source is running, a negative pressure may be built up in a pumping chamber in the housing, e.g. upon a certain rotation of the spindle moving the aggregate membrane. A reflux valve may be closed automatically then. The negative pressure may then build up in the nipple tunnel through an open milk outlet valve. Extracted milk may be drawn through a milk outlet opening in a reduction cycle of the suction means.

In an expansion cycle, in which the aggregate membrane and thus the protective membrane element can be moved towards the membrane working chamber, the milk outlet valve may close. The suction pressure within the pumping chamber may be reduced, i.e. the pressure will rise. Until a reduced threshold suction pressure may be reached within the nipple tunnel, the reflux valve may remain open. Thus, a negative pressure of about 20 mm Hg may be maintained as a minimum suction pressure within the nipple tunnel.

At appropriate pressure difference, milk may flow through a container valve, which shall be a one-way valve, into the milk chamber in the milk container.

In a reduction cycle, in which the aggregate membrane and thus the protective membrane element may be moved towards the drive housing, the suction force within the pumping chamber may be increased.

Eventually, the container valve as a one-way valve may close to prevent reflux of milk from the milk chamber into the pumping chamber. The milk outlet valve may open to allow milk to flow from the nipple tunnel into the pumping chamber, which pumping chamber may essentially be completely filled with milk.

Thus, the level of extracted milk within the milk chamber can build up during operation of the breast pump while the nipple tunnel may be filled with milk and the nipple may also constantly be provided within a negative pressure environment.

According to another solution of the invention there is provided a computer program code to run on a/the second control device which is a hand held device such as a smartphone, the computer program code designed to perform the method as described herein.

As such, another solution according to the invention is suggested in a second control device such as a smartphone which runs the computer program code as described herein.

In particular, the computer program code may comprise or consist of means to install a software, e.g. on the first/second control device. Alternatively or additionally the computer program code may comprise or consist of a software, e.g. on the first/second control device. The first/second control device may run the software and/or the computer program code. The computer program code may be understood as an app and/or a software on a smartphone which may make use of the physical components of the smartphone, such as the CPU, GPU, RAM, bluetooth device, wifi device, etc.

In particular, a/the computer program code to run on a/the second control device which is a hand held device such as a smartphone is provided, wherein the computer program code may be designed to perform the method described herein and/or may be designed to at least provide means necessary to perform the method described herein. This may be conducted by providing a visualized control panel on the second control device, e.g. due to the computer program code running on the second control device. The computer program code as well particularly enables the second control device:
to provide at least two control presets each including a control parameter,
to select one control preset as the selected control preset, and
to send the control parameter of the selected control preset to the first control device.

The computer program code may as well enable that the selected control preset may be selected through a position of a throttle which is slidable on a surface of the visualized control panel to define the position of the throttle. So the method according to the computer program code may be conducted autonomously on the second control device or with an end user input regarding moving the throttle which affects the sending step.

This facilitates retrofit and compatibility with different second control devices.

The first control device may be integrated in the breast pump. While the first control device particularly controls the suction source—e.g. control parameters, times, etc.—the first control device may be controlled by means of at least one button on the housing which the end user may press, for example to select control presets and/or control parameters. The first control device may as well be controlled by means of the second control device. The first control device may be built to receive control parameters from the second control device, in particular affecting the control of the suction source and/or to send pump data to the second control device.

The breast pump may comprise direct installation means such as a/the computer program code which can be executed on the second control device, in particular a smartphone.

The breast pump may comprise indirect installation means such as a QR code, a link or a direction to a source for acquiring a/the computer program code to be executed on the second control device, in particular a smartphone.

The computer program code may enable the second control device to provide any of the features mentioned herein related to the second control device, in particular to provide the visualized control panel, the surface of the visualized control panel, the slidable throttle, the continuous control mode, a function to record pump data, a function to store pump data, etc.

There may be provided a set with the breast pump and at least one of: the second control device the installation means and/or the computer program code.

Further details, advantages or features of the present invention will become apparent from the following description of a specific embodiment in combination with the drawings. In the drawings:

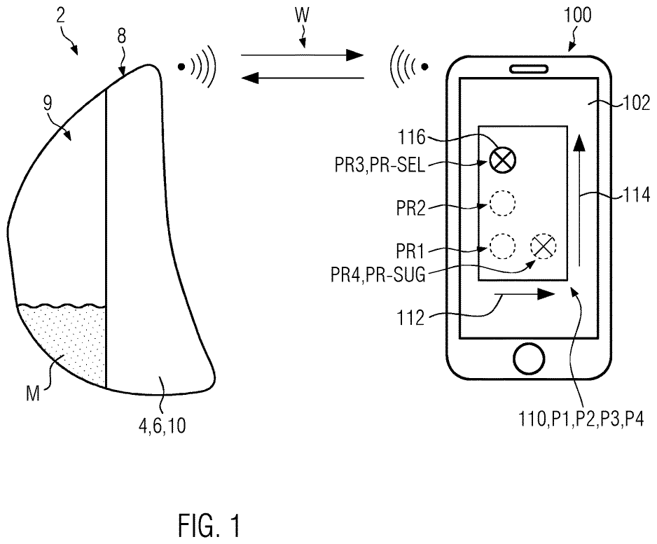

FIG. 1 is an exploded view of the individual components and elements of an embodiment of the breast pump according to the present invention; and

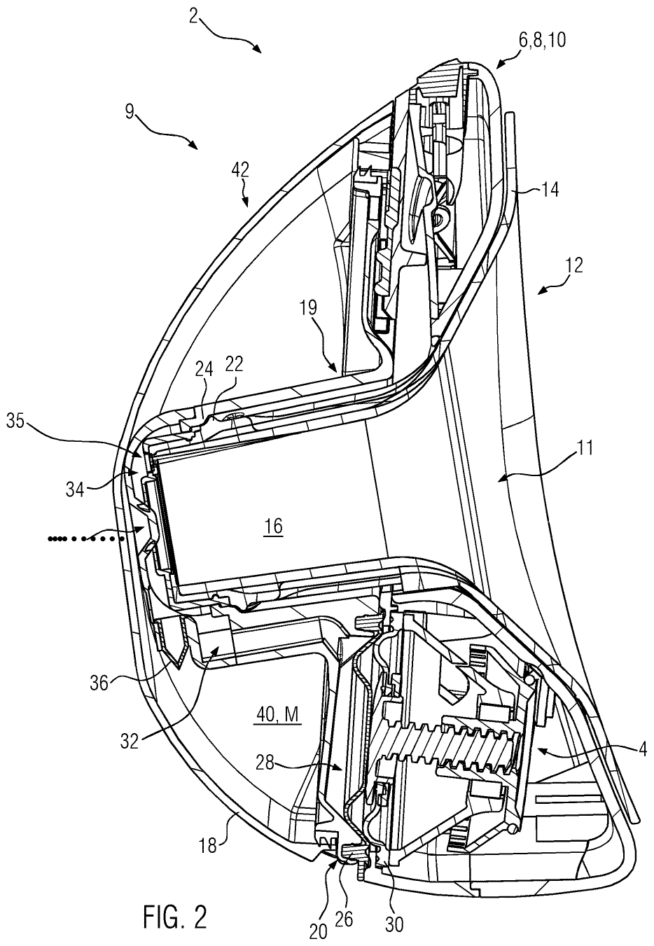

FIG. 2 is a cross-sectional view of the breast pump.

In FIG. 1 a breast pump 2 is illustrated that is wirelessly connected to a second control device 100 in the form of a smartphone and/or a hand held device separate from the breast pump 2. In particular, a first control device 10 is connected via connection means 6 to the second control device 100. The connection means 6 form(s) part of a Bluetooth data connection. The second control device 100 is as well connected to the internet, in particular a cloud service.

The breast pump 2 comprises a suction source 4 that is contained inside a housing 8 of the breast pump 2. The suction source 4 may provide a vacuum to express milk M. The first control device can control the suction source 4. The first control device 10 is built to receive control parameters P1, P2, P3, P4 from the second control device 100, each of which affect the control of the suction source 4, and is built to send pump data to the second control device 100.

Detachably supported by the housing 8 is a milk container 9 comprising a certain amount of expressed milk M. It can be seen that the milk container 9 is attached to the housing 8 so that the breast pump 2 may fit into a bra. The breast pump 2 further contains a breast shield element 12 with a nipple tunnel 16 which is not shown in FIG. 1, but in FIG. 2.

On the second control device 100 of FIG. 1 there are four different control presets PR1, PR2, PR3, PR4 provided. Each of the control presets PR1, PR2, PR3, PR4 includes and corresponds to a control parameter P1, P2, P3, P4 each of which is intended for control of the first control device 10. In other words and for example, the control parameter P1, P2, P3, P4, if sent to the first control device 10, may result in that the first control device 10 makes the suction source 4 to slow down or to be shut down.

On the second control device 100 a computer program code has been executed and/or run in order to provide the ability to perform the method described herein, in particular to control the first control device 10 by means of the second control device 10. The computer program code was downloaded from a cloud service such as an app store. By means of the computer program code, the second control device is enabled to communicate with the first control device via its connection means 6, for example to request pump data from the first control device 10, to record the pump data as recorded pump data, to store the recorded pump data on the hardware of the second control device 100, and so on.

As such, the second control device 100 runs a computer program code, i.e. in the form of an app on the smartphone. This enables the second control device 100 to visualize a visualized control panel 110 and to control the first control device 10.

The second control device 100 has a display 102 that visualizes the control panel 110. On the surface of the control panel 110 a throttle 116 is movable along a first dimension 112 and a second dimension 114 in order to slide the throttle 116 from one to another position. The position of the throttle 116 may select one particular control preset PR1, PR2, PR3, PR4 from the four control presets PR1, PR2, PR3, PR4. In the illustration, the control preset PR3 is also the selected control preset PR-SEL which is highlighted as opposed to the other control presets PR1, PR2. Along the second dimension 114 the throttle 116 may alternatively occupy the positions at the control presets PR2 and PR1. Upon moving/sliding the throttle 116 along the first dimension 112 and the second dimension 114 the position at control preset PR4 may be chosen from the given ones.

In the given visualization of the control panel 110, the control preset PR4 is the suggested control preset PR-SUG. This is visualized by highlighting as opposed to the selected control preset PR-SEL and the other control presets PR1 and PR2. The control preset PR4/PR-SUG is thus for example provided with a central cross so that the end user may see where the suggested control preset PR-SUG easily.

In the case presented here, the second control device 100 may send the control parameter P1, P2, P3, P4 that corresponds to the selected control preset PR-SEL to the first control device 10 which then controls the first control device by means of this control parameter P1, P2, P3, P4. The sending occurs at least each time the throttle 116 is moved, but the sending may alternatively or additionally occur once or more times per second, in particular independently from moving of the throttle.

On the second control device 100 there is provided a continuous control mode that not only may not only suggest the suggested control preset PR-SUG, but as well may autonomously select different control presets PR1, PR2, PR3, PR4 over time. This may override the position of the throttle 116 an end user selects. The continuous control mode may be activated and deactivated by the end user by an interaction on the surface of the visualized control panel 110. For example, the continuous control mode may have a stimulation mode active for one or more minutes and then may have an expression mode active for several minutes.

The second control device 100 is able to record pump data as recorded pump data when the breast pump 2 is running. The pump data is transmitted via the wireless connection W and stored on the second control device.

The suggested control preset PR-SUG has been determined based on the recorded pump data. In this regard, the second control device 100 is able to draw conclusions from the recorded pump data in order to derive a suggestions what fits best to the end user.

In FIG. 2 the breast pump 2 of FIG. 1 is illustrated in a cross section and at a higher level of detail. It can be seen that the breast pump 2 has a housing 8 that contains the suction source 4, the connection means 6 and the first control device 10.

The suction source 4 is of a membrane pump type which is essentially fully integrated into the housing 8.

In this embodiment the vacuum/suction can be physically generated within the housing 8. Not shown is that the housing 8 holds a rechargeable battery/accumulator adapted to energize the suction source 4 and the first control device 10.

The suction source 4 has a protective membrane element 26 to cover an aggregate membrane 30 that can be moved by turning a spindle. When the aggregate membrane 30 is moved, a membrane working chamber 28 in front of the protective membrane element 26 can be changed in its volume to have the suction function.

Here, the housing 8 is made of opaque plastic material. In a side view, the housing is wedge-shaped. A bottom section of the housing 8 is considerably thicker than a top section of the housing 8. In a front or rear view, the housing is essentially avocado-shaped and/or round. The housing 8 circumferentially encloses a bore 11 to insert the breast shield element 12.

Detachably attached to the housing 8 is the milk container 9.

The milk container 9 has a milk chamber 40.

Inserted through the bore 11 of the housing 8 a nipple tunnel 16 of the breast shield element 12 is connected to the milk container 9 via a thread 22 to a thread 24 of the milk container 9. In other words, the milk container is provided with the thread 24 cooperating with the thread 22 of the nipple tunnel 28 to secure the breast shield element 24 against the milk container 36, especially when the nipple tunnel 28 projects through the bore 11 of the housing 2.

A flange 14 of the breast shield element 12 shall receive a nipple and an areola.

The milk container 9 comprises a container shell element 18 and a lid element 19 closing the container shell element 18. The lid element 19 is connected with the spherical container shell element 18 by means of a hinge 20. Both the lid element 19 and the spherical container shell element 18 provide surfaces defining the hinge 20. Cooperating surfaces of the hinge 20 allow pivoting of the lid element 19 toward the container shell element 18.

For operation of the breast pump 2 of FIG. 2, the nipple shall be arranged within the nipple tunnel 28. Proper alignment of the nipple is observable through a transparent window 42 in the milk container 9 from above.

It is possible that the suction source 4 is being activated, for example upon pressing a button e.g. above on the housing 9 giving a command to the first control device 10 which then controls the suction source 4 and upon an interaction on the second control device 100 by the end user which then sends a control parameter P1, P2, P3, P4 to the first control device which then controls the suction source 4.

When the suction source 4 is running, a negative pressure is built up in a pumping chamber 32 in the housing 8 upon a certain rotation of the spindle moving the aggregate membrane 30. A reflux valve 38 is closed. The negative pressure will build up in the nipple tunnel 16 through an open milk outlet valve 34. Extracted milk M is drawn through a milk outlet opening 35 in a reduction cycle of the suction means 4.

In an expansion cycle, in which the aggregate membrane 30 and thus the protective membrane element 26 is moved towards the membrane working chamber 28, the milk outlet valve 34 will close. The suction pressure within the pumping chamber 32 will be reduced, i.e. the pressure will rise. Until a reduced threshold suction pressure is reached within the nipple tunnel 16, the reflux valve 38 will remain open. Thus, a negative pressure of about 20 mm Hg will be maintained as a minimum suction pressure within the nipple tunnel 16. At appropriate pressure difference, milk M will flow through a container valve 36, which is a one-way valve, into the milk chamber 40 in the milk container 9.

In a reduction cycle, in which the aggregate membrane 30 and thus the protective membrane element 26 is moved towards the drive housing 70, the suction force within the pumping chamber 32 is increased.

Eventually, the container valve 36 as a one-way valve will close to prevent reflux of milk M from the milk chamber 40 into the pumping chamber 32. The milk outlet valve 34 will open to allow milk M to flow from the nipple tunnel 16 into the pumping chamber 32, which pumping chamber 32 is essentially completely filled with milk M.

Thus, the level of extracted milk M within the milk chamber 40 builds up during operation of the pump while the nipple tunnel 16 is filled with milk M and the nipple is constantly provided within a negative pressure environment.

### REFERENCES

2 breast pump
4 suction source (e.g. vacuum pump or vacuum pump connector!)
6 connection means (e. g wireless connection module)
8 housing
9 milk container
10 first control device
11 bore
12 breast shield element
14 flange of the breast shield element
16 nipple tunnel
18 container shell element
19 lid element
20 hinge
22 thread
24 thread
26 protective membrane element
28 membrane working chamber
30 aggregate membrane
32 pumping chamber
34 milk outlet valve
35 milk outlet opening
36 container valve
38 reflux valve
40 milk chamber
42 window
100 second control device
102 display
110 visualized control panel
112 first dimension
114 second dimension
116 throttle
PR1 control preset
PR2 control preset
PR3 control preset
PR4 control preset
PR-SEL selected control preset
PR-SUG suggested control preset
M expressed milk
P1 control parameter of PR1
P2 control parameter of PR2
P3 control parameter of PR3
P4 control parameter of PR4
W wireless connection between 10 and 100

1. A method to control a breast pump having a suction source, connection means and a first control device, wherein the first control device is adapted to control the suction source, and wherein the first control device is connected via the connection means to a second control device which is a hand held device separate from the breast pump, the method including:
providing on the second control device at least two control presets (PR1, PR2, PR3, PR4), each of the at least two control presets (PR1, PR2, PR3, PR4) including one of at least two control parameters (P1, P2, P3) intended for control of the first control device,
selecting on the second control device one control preset (PR1, PR2, PR3, PR4) of the at least two control presets (PR1, PR2, PR3, PR4) as the selected control preset (PR-SEL),
sending by means of the second control device the control parameter (P1, P2, P3, P4) of the selected control preset (PR-SEL) to the first control device and controlling the first control device by means of said control parameter (P1, P2, P3, P4),
wherein the selected control preset (PR-SEL) is selected through a position of a throttle which is slidable on a surface of a visualized control panel of the second control device to define the position of the throttle

2. The method according to claim 1, the control panel having two dimensions and the throttle being movable along the one or two dimensions to define the position of the throttle.

3. The method according to claim 1, at least one of the selected control preset (PR-SEL) or the position of the throttle being further selected through a continuous control mode acting on the second control device, the continuous control mode being able to select different control presets (PR1, PR2, PR3, PR4) over time.

4. The method according to claim 1, the at least two control parameters (P1, P2, P3) including at least one of a group including a pump duration, pump amplitude, a pump on-time, a pump off-time, a pump frequency, a milk volume, a number of pump sessions, and a position of the breast pump.

5. The method according to claim 1, further including providing on the second control device a suggested control preset (PR-SUG) as one of the at least two control presets (PR1, PR2, PR3, PR4) and visualizing the suggested control preset (PR-SUG) on the second control device (100), (116).

6. The method according to claim 5, wherein the suggested control preset (PR-SUG) is provided by one of the first control device or the second control device control mode.

7. The method according to claim 1, further comprising recording of pump data as recorded pump data when the breast pump is running, wherein the recorded pump data includes at least one of a pump duration, pump amplitude, a pump on-time, a pump off-time, a pump frequency, a milk volume, a number of pump sessions, and a position of the breast pump.

8. The method according to claim 7, the recording being conducted by the second control device

9. The method according to claim 6, the suggested control preset (PR-SUG) being determined based on the recorded pump data.

10. The breast pump to perform the method of claim 1, having a suction source, connection means and a first control device, wherein the first control device is adapted to control the suction source, and wherein the first control device is adapted to be connected via the connection means to a second control device which is a hand held device separate from the breast pump.

11. The breast pump according to claim 10, wherein the first control device is built to receive control parameters (P1, P2, P3, P4) from the second control device affecting the control of the suction source and to send pump data to the second control device.

12. The breast pump according to claim 10, further comprising a housing and a milk container detachably supported by the housing, the housing holding at least one of a group including the suction source, the connection means and/or the first control device.

13. A computer program code to run on a second control device which is a hand held device that is a non-transitory computer readable medium the computer program code designed to perform the method of one of claim 1.

14. The second control device which is a hand held device running the computer program code of claim 13.

15. The second control device of claim 14, wherein the hand held device is a smartphone.
